(19) United States
(12) Patent Application Publication
(10) Pub. No.: US 2019/0134115 A1
(43) Pub. Date: May 9, 2019

(54) Activated Lactobacillus Reuteri Strains for Selective Pathogen Inhibition in a Human Microbial Community

(71) Applicant: BioGaia AB, 64 Stockholm (SE)

(72) Inventors: Stephan Roos, Uppsala (SE); Jennifer Kristine Spinler, Pearland, TX (US)

(73) Assignee: BioGaia AB, 64 Stockholm (SE)

(21) Appl. No.: 16/095,284

(22) PCT Filed: Nov. 7, 2017
(86) PCT No.: PCT/EP2017/078463

§ 371 Date: Oct. 19, 2018

Related U.S. Application Data
(60) Provisional application No. 62/418,471, filed on Nov. 7, 2016.

(30) Foreign Application Priority Data
Nov. 7, 2016 (SE) 1651459-8

(51) Int. Cl. A61K 35/747; A01N 63/02; A61P 31/04; C12N 1/04

(52) U.S. Cl. CPC A61K 35/747; C12N 1/04; A61P 31/04; A01N 63/02

(57) ABSTRACT

Herein is disclosed a probiotic composition comprising cells of Lactobacillus reuteri preloaded with reuterin for use in the treatment or prevention of an infection caused by Clostridium difficile and/or other glycerol metabolizing pathogen(s) in an individual, and a method for the treatment or prevention of an infection caused by Clostridium difficile and/or other glycerol metabolizing pathogen(s) in an individual. Further provided is a method for manufacturing a composition comprising L. reuteri pre-loaded with reuterin, and compositions obtained by said method.)

### FIELD OF THE INVENTION

The invention relates to probiotic products, particularly oral probiotic products, based on the activation of the reuterin-production machinery of Lactobacillus reuteri, for use in patients to combat pathogens, including Clostridium difficile.

### BACKGROUND OF THE INVENTION

Rising antimicrobial resistance and dwindling new antibiotic discovery are increasing the risk of a global infectious disease healthcare crisis. In the United States, the Centers for Disease Control estimates that antibiotic resistance causes over 2 million illnesses and 23,000 deaths each year (1). An additional half a million individuals are hospitalized with antibiotic-associated Clostridium difficile infection (CDI), contributing a further 29,000 deaths (2) and a $5.4 billion burden to the U.S. health care system each year (3). While the majority of patients with a primary CDI diagnosis will respond to front-line antibiotic treatment (vancomycin or off-label metronidazole), up to 35% will experience a CDI relapse (4, 5) and a significantly increased risk for multiple disease recurrences (6). The limited treatment options for recurrent CDI include extended antibiotic regimens (7), further contributing to the cycle of reinfection. Patients who suffer several antibiotic treatment failures are often referred for fecal transplantation, an investigational therapy with unknown long-term consequences (8). The National Action Plan to Combat Antibiotic-Resistant Bacteria (9) emphasizes that efforts are needed to advance the development of new antibiotics and alternative therapies to fight resistance and diseases associated with antimicrobial use. As a result, a number of emerging therapies are being investigated, including probiotics, immunotherapies, toxin binding agents, defined microbial therapy and non-toxigenic Clostridium difficile (C. difficile) strains (7, 10).

Antibiotic disruption of a healthy microbiota leaves the host susceptible to CDI. Probiotics are a promising alternative therapy and are proposed to combat antimicrobial-associated diseases by preventing pathogen invasion and protecting the healthy microbiota. Some promising adjunct therapies include supplementing staggered antibiotic withdrawal with Lifeway Kefir in recurrent CDI cases (11), or co-administrating Bio-K+ with antibiotics to decrease primary CDI incidence (12, 13). In the U.S., adjunct therapy is not yet recommended by the Society for Healthcare Epidemiology of America or the Infectious Diseases Society of America despite a growing literature supporting probiotics use for CDI prevention (14, 15). Nevertheless, next-generation probiotics are being actively investigated for use in CDI prevention (16).

Lactic acid bacteria have long been considered important protectors of gut health, with many probiotic organisms belonging to the genus Lactobacillus. Several Lactobacillus spp.

are intrinsically resistant to antibiotics and this is an important consideration when considering probiotics as adjunct treatment to antimicrobial therapy (17). Naturally occurring antimicrobial production by host-specific probiotic bacteria is a rich area for further innovation of non-traditional therapies targeting drug-resistant pathogens. Certain human-derived probiotic L. reuteri strains produce an isomeric mixture of 3-hydroxypropionaldehyde (3-HPA) (18), a three-carbon secondary metabolite commonly known as reuterin, with broad spectrum in vitro antimicrobial activity against enteric pathogens (19). The vitamin B₁₂-dependent production of reuterin occurs when L. reuteri ferments the substrate, glycerol. This process is driven by the horizontally acquired 57-gene pdu-cbi-hem-cob cluster containing genes important for: 1) de novo vitamin B₁₂ synthesis, 2) the generation of microcompartments where reuterin production occurs, and 3) glycerol fermentation to reuterin (20-22). Indicative of its probiotic character, reuterin does not typically interfere with the growth of commensal lactic acid bacteria (19), and preliminary reports indicate that this compound may promote microbiota diversity, a potential consideration in CDI (23).

Glycerol, a common dietary component, is used as a sweetener, solvent, thickener and preservative in pharmaceutical and food products and is a natural component of triglycerides. Glycerol absorption primarily occurs in the small intestine, although unabsorbed glycerol, free fatty acids, and undigested glycerides do transit to the colon. Gut microbes produce bacterial lipases that hydrolyze glycerides into free fatty acids and glycerol (73). Other sources of colonic glycerol are luminal fat digestion, intestinal clearing of endogenous plasma glycerol, desquamated epithelial cells and luminal microbial fermentation (19). Microbial fermentation of glycerol can occur by organisms in the class Clostridia and families Enterobacteriaceae and Lactobacillaceae, and can result in many products including butyrate or 1,3-propanediol that can either stimulate or selectively inhibit glycerol absorption in the colon (57, 74, 75).

Hence, as glycerol is also a substrate and/or electron acceptor for C. difficile and many other pathogen species as described above it would not be wise to add glycerol to the intestine together with a probiotic product (intended to produce and release reuterin) as that would create a risk of favouring certain pathogens, including stimulating growth of such bacteria already in the intestine.

Accordingly, there is still a need in the art for identifying alternative and/or improved treatments for infections in individuals caused by Clostridium difficile and/or other glycerol-metabolizing pathogens, and particularly for treatments that can be used before, in combination with and/or after treating an individual with antibiotics.

### SUMMARY OF THE INVENTION

The problem described above is solved or at least mitigated by the present invention disclosed herein.

Accordingly, herein is provided an oral composition comprising cells of Lactobacillus reuteri (L. reuteri) pre-loaded with reuterin for use in the treatment or prevention of an infection caused by Clostridium difficile and/or other glycerol metabolizing pathogen(s), e.g. other glycerol metabolizing pathogenic bacteria, in an individual. The oral composition is preferably in the form of a tablet or a capsule. The treatment or prevention of the infection is accomplished by orally administering the L. reuteri comprising reuterin loaded microcompartments to the individual.

In a particular embodiment, the treatment or prevention of the infection is accomplished without adding glycerol to the gastrointestinal tract, i.e. without administering glycerol to the gastrointestinal tract in addition to the oral composition comprising cells of L. reuteri. In other words, the treatment or prevention of the infection caused by Clostridium difficile and/or other glycerol metabolizing pathogen(s) may be accomplished without administering glycerol in connection with administering the oral composition comprising cells of L. reuteri. Here, “in connection with” is to be understood as meaning in combination with, or together with, administering the oral composition comprising cells of L. reuteri. An advantage of not administering glycerol in conjunction to administering the oral composition comprising cells of L. reuteri is that the Clostridium difficile and/or other pathogenic bacteria do not get access to glycerol, which could be metabolized by the pathogenic bacteria and which thereby would promote growth of the pathogenic bacteria. For the same reason, it is advantageous not to add any glycerol to the oral composition comprising the cells of L. reuteri before administration to an individual.

It is however essential to deliver the oral composition in a way that the reuterin from the microcompartments is not released until it reaches the gastrointestinal tract, such that the L. reuteri accomplish secretion of reuterin without the need for further glycerol metabolization and hence can be used as an adjunct probiotic therapy for CDI in the gastrointestinal tract. This is achieved for example if the oral composition is in the form of a tablet or a capsule, preferably a tablet having a protective coating or an enteric coated capsule.

In another embodiment, the composition comprising cells of L. reuteri is to be administered to said individual before, in combination with, and/or after a treatment with one or more antibiotic(s) effective against an infection caused by Clostridium difficile in said individual. Here, it is to be understood that it would be suitable to administer a composition comprising L. reuteri as long as it is suspected that pathogenic bacteria, including C. difficile, are present in the individual.

There is also provided herein a method for the treatment or prevention of an infection caused by Clostridium difficile and/or other glycerol metabolizing pathogen(s), e.g. other glycerol metabolizing pathogenic bacteria, in an individual, said method comprising administering a composition comprising Lactobacillus reuteri (L. reuteri) cells pre-loaded with reuterin in a pharmaceutically effective amount to said individual.

Herein is further provided a method for manufacturing a composition comprising L. reuteri pre-loaded with reuterin, said method comprising the steps of:

a) freeze-drying cells of L. reuteri, and b) pre-incubating the freeze-dried cells of L. reuteri in glycerol, alternatively in glycerol and a carbon source, such as lactose or dextrose, and yeast extract. The glycerol, alternatively a mixture of glycerol, a carbon source, such as lactose or dextrose, and yeast extract, is/are added to the cells after the freeze-drying step but prior to for example dosing.

Also provided herein is a composition obtained by the above-described method. The composition may be provided for use in the treatment or prevention of an infection caused by Clostridium difficile and/or other glycerol metabolizing pathogen(s), e.g. other glycerol metabolizing pathogenic bacteria, in an individual.

As previously described in European Patent EP2303295 B1, pre-loading of reuterin may alternatively be performed by priming the reuterin producing machinery with glycerol or 1,2-propanediol (1,2-PD) and later in the manufacturing process of cell-cultures adding glycerol to the bacterial culture, at a certain point before preservation. The pre-loading of reuterin may further comprise the addition of vitamin B12, cobalt and/or Vitamin C to the growth media for improving the conditions for optimal growth and production of microcompartments and reuterin of L. reuteri bacteria during the manufacturing process.

Herein is further provided a method for manufacturing an oral composition comprising L. reuteri pre-loaded with reuterin, said method comprising the steps of:

a) freeze-drying cells of L. reuteri pre-loaded with reuterin, and b) distributing the freeze-dried cells into capsules, or making (i.e. manufacturing or producing) tablets comprising the freeze-dried cells. In an embodiment, the tablet has a protective coating. In another embodiment, the capsule is an enteric coated capsule. It is advantageous not to add glycerol to the oral composition comprising L. reuteri pre-loaded with reuterin during and/or after this method for manufacturing an oral composition. In an embodiment, the L. reuteri pre-loaded with reuterin, which are to be freeze-dried, have previously been manufactured according to the method for manufacturing a composition comprising L. reuteri pre-loaded with reuterin as described above.

Also provided herein is an oral composition obtained by the above-described method for manufacturing an oral composition comprising L. reuteri pre-loaded with reuterin. The composition may be provided for use in the treatment or prevention of an infection caused by Clostridium difficile and/or other glycerol metabolizing pathogen(s), e.g. other glycerol metabolizing pathogenic bacteria, in an individual.

In the intestine the pre-loaded L. reuteri will release reuterin and will further also compete with C. difficile for available glycerol.

In this respect, before explaining some embodiments of the invention in detail, it is to be understood that the invention is not limited in its application to the details of construction and to the arrangements of the components set forth in the following descriptions or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

FIG. 1. Zones of inhibition show L. reuteri inhibits C. difficile in a strain-specific and reuterin-dependent manner in vitro. L. reuteri strains or a vancomycin disc (5 μg) were developed on BHI+20 mM glucose. C. difficile strains were overlain in a BHI soft agar containing 2% glycerol and incubated for growth. Clear zones of inhibition were measured (mm). The picture shows a bar graph representing zone of inhibition measurements (mm) for all strains tested. All results represent the mean±S.E.M, n=3 Inhibitory zones significantly larger (unpaired, 2-tailed t test with equal variances) than either the corresponding vancomycin control (a, p-value<0.05), the 6475 WT zones (b, p-value<0.05), or the 6475: :pocR (c, p-value<0.05) are indicated.

FIG. 2. Precursor-directed reuterin production by L. reuteri suppresses growth of C. difficile in a human fecal microbial community.

A) Timeline depicting experimental design of MBRA experiments. B) Quantities of 16S rRNA gene copies of C. difficile relative to total 16S rRNA gene copies in bioreactor samples over time. Data are represented as the mean±S.D. Significant differences between the Ctrl and Lreu-Glyc groups in relative C. difficile 16S rRNA gene copies over seven time points is indicated (unpaired, 2-tailed t test with unequal variances).

FIG. 3. Co-delivery of L. reuteri and glycerol diminished symptoms of CDI in vivo. A) Experimental timeline of the mouse model of recurrent C. difficile infection (n=5 mice per group). B) C. difficile concentrations in stool from day 2 were measured by CFU counts on CCFA media. C) TcdA-mediated cytotoxicity in mouse feces from day 2 was measured by cell rounding of CHO (Chinese Hamster Ovary) cells under a phase-contrast microscope and expressed as the reciprocal of the highest dilution that resulted in >80% cell rounding per gram of feces. D) Histopathology damage scores of C. difficile infected, glycerol only (Glyc), C. difficile infected, L. reuteri DSM 17938::pocR and glycerol treated (Lreu-pocR), and C.

difficile infected, L. reuteri DSM 17938 WT and glycerol treated (Lreu-Glyc) groups. Data are represented as means±S.E.M. Significant differences (p<0.05), as determined using an unpaired, two-tailed t test with unequal variances, are indicated by the placement of different letters above each category column.

FIG. 4. Loaded L. reuteri DSM 17938 suppresses growth of C. difficile CD2015 in vitro. Quantities of 16S rRNA gene copies of C. difficile CD2015 relative to total 16S rRNA gene copies in co-culture samples comparing dose of L. reuteri DSM 17938 wild-type and gdh mutant (17938::gdh) strains. Data are represented as the mean±S.E.M.

### DETAILED DESCRIPTION OF THE INVENTION

Herein, the survival of select probiotic candidates under antibiotic pressure was investigated. Using drugs marketed for treating C. difficile infection (CDI), our screening highlighted L. reuteri as the least susceptible to antibiotics. Further analysis showed that L. reuteri production of reuterin through fermentation of glycerol prohibited C. difficile invasion of antibiotic-disrupted microbial community models, making this precursor-directed antimicrobial biosynthesis strategy a front-runner for customized probiotics to be used alone or as adjunct therapy, i.e. an assisting treatment used together with a primary treatment, in CDI prevention.

Generally, the manufacture of L. reuteri cultures to be used as probiotics are cultured in the absence of glycerol and thereafter lyophilized. In those bacteria, the machinery used for reuterin production have not been activated, but under favorable conditions the system can be active in 5-60 minutes after the bacteria come in contact with glycerol and other needed components as described herein.

The L. reuteri culture can be loaded with reuterin, according to the description herein by the presence of glycerol during the manufacture of the culture or by a pre-incubation step of glycerol prior to dosing. The glycerol (final concentration 1-500 mM) can be added during the fermentation step, it can be added together with cryo-protectants at the step after fermentation and possible washing, but before freeze-drying, or it can be added as a pre-incubation step of the freeze-dried bacteria prior to dosing. The reuterin production machinery including the formation of microcompartments of L. reuteri can be improved by priming the reuterin producing machinery with 1,2-PD or glycerol at the start of the fermentation.

The cell-culture product can be manufactured in several ways, including but not limited to the three different ways below:

1) L. reuteri cells are allowed to convert glycerol into reuterin at the end of the fermentation step of the manufacturing process but before the freeze-drying step. A product prepared in this way will contain freeze-dried cells and reuterin both within and surrounding the cells. With this manufacture design the freeze-dried bacteria are loaded with reuterin.

2) Like 1 but the reuterin-production machinery of the bacteria is primed with 1,2-PD or glycerol and possibly cobalt or vitamin B12 at the start of the fermentation step. With this manufacture design the freeze-dried bacteria are loaded with both reuterin and are primed with the capacity to make and store reuterin.

3) L. reuteri cells are allowed to convert glycerol into reuterin after the fermentation and possible washing step, with the addition of glycerol and then allowing for the reuterin production around 30-45 minutes at 37° C. prior to the freeze-drying step. The addition of glycerol for the formation of reuterin can for example be made together with the cryo-protectants. The reuterin-production machinery of the bacteria is primed with 1,2-PD or glycerol at the start of the fermentation step. Advantages of the manufacturing design 3 in relation to way 2 are that way 3 is better suited to be used in many industrial manufacturing set-ups and may allow for better control of the reuterin formation.

Another way of preparing the product would be to produce the freeze-dried bacteria according to standard manufacturing processes (no addition of glycerol) and then having the freeze-dried bacterial product pre-incubated in glycerol prior to dosing. The pre-incubation step can be done for about 5-60 mins.

Addition of 1,2-PD or glycerol to the growth media has effects both on survival and microcompartments (MCS) formation. The enzyme complex PduCDE responsible for the conversion of 1,2-PD into propionaldehyde, are also responsible for the conversion of glycerol into reuterin, which opens the possibility that the MCS formed when bacteria are grown in the presence of 1,2-PD can also work as factories for production of reuterin if the bacteria come in contact with glycerol and lacks means for further metabolism of reuterin (i.e. the bacteria are in stationary phase or are exposed to glycerol in a water solution). The reuterin formed in the MCS are retained within the cell in a higher amount compared to a cell that lacks MCS. This enables the bacteria to be “loaded” with reuterin prior to i.e. freeze-drying.

Apart from priming with glycerol and 1,2-PD, addition of certain other substances to the growth media showed to have effects on survival of cells, formation of MCS, production of reuterin and fitness of the bacteria, those substances are for example Vitamin B12, cobalt and Vitamin C.

The preserved L. reuteri loaded with activated MCS containing reuterin can be formulated into various compositions, typically enteric capsules, tablets, oils, powders and the like. Such formulations can be prepared by known means, using pharmaceutically acceptable carriers, excipient, solvents or adjutants. Such procedures and ingredients are well known and amply described in standard texts and manuals.

A composition according to the invention can be used for example for the prophylaxis (prevention) or the treatment of disorders linked to pathogens that are able to use glycerol as a substrate or electron acceptor, such as C. difficile, Clostridium perfringens, Clostridium botulinum, Fusobacterium nucleatum, Escherichia coli, Shigella spp, Salmonella enterica, Klebsiella spp., Citrobacter freundii, Yersinia enterocolitica, Aeromonas hydrophile and Listeria monocytogenes. The composition can be given alone or in combination with a suitable antibiotic such as vancomycin, metronidazole, and/or fidaxomycin.

Herein, it is demonstrated that for example L. reuteri DSM 17938, a probiotic strain clinically indicated to reduce viral and bacterial diarrhea in children (58-60) and respiratory and GI disease in adults (61, 62), works as preventative therapy in CDI (C. difficile Infection) The delivery of pre-loaded L. reuteri prevented C. difficile from establishing high concentrations in a number of antibiotic-treated microbial communities, and the suppression of C. difficile growth remained after terminating L. reuteri supplementation.

Additionally, compositions comprising pre-loaded L. reuteri and glycerol, according to the invention, decreased TcdA toxicity in feces, and resulted in less cecal pathology in a mouse model of recurrent disease.

Accordingly, there is provided herein a composition comprising cells of Lactobacillus reuteri (L. reuteri) pre-loaded with reuterin for use in the treatment or prevention of an infection caused by Clostridium difficile and/or other glycerol metabolizing pathogen(s) in an individual. A glycerol metabolizing pathogen(s) refers to a pathogenic microorganism, i.e. a pathogen, herein more specifically a bacterium, which e.g. uses glycogen as a source of energy, e.g. bacteria of the species C. difficile, Clostridium perfringens, Clostridium botulinum, Fusobacterium nucleatum, Escherichia coli, Shigella spp, Salmonella enterica, Klebsiella spp., Citrobacter freundii, Yersinia enterocolitica, Aeromonas hydrophila and Listeria monocytogenes. Hence, there is provided herein a composition for use wherein the term “other glycerol metabolizing pathogen(s)” is defined as being a glycerol metabolizing pathogen(s) selected from the group consisting of bacteria of the species Clostridium perfringens, Clostridium botulinum, Fusobacterium nucleatum, Escherichia coli, Shigella spp, Salmonella enterica, Klebsiella spp., Citrobacter freundii, Yersinia enterocolitica, Aeromonas hydrophila and Listeria monocytogenes. There is provided a composition for use in the treatment or prevention of an infection caused by Clostridium difficile and/or other glycerol metabolizing pathogen(s), e.g. other glycerol metabolizing pathogenic bacteria, in an individual, wherein said composition comprises L. reuteri DSM 17938.

Herein, the term “individual” encompasses an animal, including a human.

A composition for use as disclosed herein may be administered to said individual before, in combination with, and/or after a treatment with one or more antibiotic(s) effective against an infection caused by Clostridium difficile and/or other glycerol metabolizing pathogen(s) in said individual. As described above, said other glycerol metabolizing pathogen(s) are selected from the group consisting of bacteria of the species Clostridium perfringens, Clostridium botulinum, Fusobacterium nucleatum, Escherichia coli, Shigella spp, Salmonella enterica, Klebsiella spp., Citrobacter freundii, Yersinia enterocolitica, Aeromonas hydrophila and Listeria monocytogenes.

An oral composition as disclosed herein may be provided in a pharmaceutically effective amount to said individual. The composition of the present invention, comprising the pre loaded L. reuteri, is administered orally in a way that it is not released in the oral cavity as the reuterin is very active and would then not reach the needed site of the C. diff infection. In one embodiment the composition is in the form of a tablet with a protective coating or a capsule (e.g. a hard capsule such as a capsule made of gelatin or polymers (e.g. HPMC)). Gelatin capsules include but are not limited to Coni-Snap (Capsugel). In a particular embodiment administration of the pre loaded L. reuteri is thus achieved without adding glycerol to the gastrointestinal tract. In a particular embodiment the composition is an enteric coated capsule. In particular embodiments of the invention the enteric coated capsules comprise cellulosic derivatives. The enteric coated capsules include but are not limited to VCAPS enteric capsules (Capsugel).

Examples of polymers for protective coatings of tablets include, but are not limited to, Kollicoat® Protect (polyvinyl alcohol-polyethylene glycol copolymer and polyvinyl alcohol, BASF®), Kollicoat® Smartseal 30 D (methyl methacrylate (MMA) and diethylaminoethyl methacrylate), Opadry® amb II (Colorcon®), Eudragit® E 100, Eudragit® E 12.5, Eudragit® E PO, Hydroxypropylmethylcellulose (e.g. Methocel®, Anycoat®, Pharmacoat®), Hydroxypropylcellulose (e.g. Coatcel® and Klucel®), Hydroxyethylcellulose (e.g. Natrosol®), poly (vinyl pyrrolidone) (e.g. Kollidon®), poly (vinyl pyrrolidone)/poly (vinyl acetate) copolymers, poly (vinyl alcohol)/poly (ethylene glycol) copolymers (e.g. Kollicoat® IR), poly (ethylene glycol), maltodextrines and polydextrose.

Accordingly, herein is provided a composition comprising cells of Lactobacillus reuteri (L. reuteri) pre-loaded with reuterin for oral use in the treatment or prevention of an infection caused by Clostridium difficile and/or other glycerol metabolizing pathogen(s) in an individual. The treatment or prevention of the infection is accomplished by orally administering the L. reuteri comprising reuterin loaded microcompartments to the individual. It is however essential to deliver the composition in a way that the reuterin from the microcompartments is not released until it reaches the gastrointestinal tract, thus the L. reuteri accomplish secretion of reuterin without the need for further glycerol metabolization and hence can be used as an adjunct probiotic therapy for CDI in the gastrointestinal tract.

There is in another aspect provided herein a method for the manufacture of a composition comprising L. reuteri pre-loaded with reuterin, comprising the steps of: a) freeze-drying cells of L. reuteri, and b) preincubating the freeze-dried cells of L. reuteri with glycerol. Step b) of such a method may be performed for about 5 to 60 minutes.

Furthermore, in step b) the freeze-dried cells of L. reuteri may be preincubated with a mixture of glycerol, a carbon source, such as lactose or dextrose, and yeast extract.

There is also provided a composition obtained by the above method for manufacture, for use in the treatment or prevention of an infection caused by Clostridium difficile and/or other glycerol metabolizing pathogen(s) in an individual. Said composition may further comprise an antibiotic(s) effective against an infection caused by Clostridium difficile and/or other glycerol metabolizing pathogen(s). Such an antibiotic may for example be selected from vancomycin, metronidazole, and/or fidaxomycin.

Herein is further provided a method for manufacturing an oral composition comprising L. reuteri pre-loaded with reuterin, said method comprising the steps of:

a) freeze-drying cells of L. reuteri pre-loaded with reuterin, and b) distributing the freeze-dried cells into capsules, or making tablets comprising the freeze-dried cells.

Also provided herein is an oral composition obtained by the above-described method for manufacturing an oral composition comprising L. reuteri pre-loaded with reuterin. The composition may be provided for use in the treatment or prevention of an infection caused by Clostridium difficile and/or other glycerol metabolizing pathogen(s), e.g. other glycerol metabolizing pathogenic bacteria, in an individual.

In yet another aspect, there is provided a method for the treatment or prevention of an infection caused by Clostridium difficile and/or other glycerol metabolizing pathogen(s) in an individual, said method comprising administering a composition comprising Lactobacillus reuteri (L. reuteri) cells pre-loaded with reuterin in a pharmaceutically effective amount to said individual. Said composition may be administered to said individual before, in combination with, and/or after a treatment with one or more antibiotic(s) effective against an infection caused by Clostridium difficile and/or other glycerol metabolizing pathogen(s) in said individual. Such method for the treatment or prevention of an infection caused by Clostridium difficile and/or other glycerol metabolizing pathogen(s) in an individual comprises using any composition as mentioned herein. As described above, said other glycerol metabolizing pathogen(s) are selected from the group consisting of bacteria of the species Clostridium perfringens, Clostridium botulinum, Fusobacterium nucleatum, Escherichia coli, Shigella spp, Salmonella enterica, Klebsiella spp., Citrobacter freundii, Yersinia enterocolitica, Aeromonas hydrophile and Listeria monocytogenes.

In summary, our results show that probiotic L. reuteri DSM 17938 is intrinsically resistant to antimicrobial drugs used to treat antibiotic-associated CDI and has reuterin-mediated antimicrobial activity against C. difficile. Delivery of pre-loaded L. reuteri DSM 17938 interferes with C. difficile growth within an antibiotic-treated human-fecal microbial community in vitro without significantly affecting the overall microbial community composition. Additionally in an animal model of recurrent CDI, we provide evidence that delivery of pre-loaded L. reuteri DSM 17938 reduce the toxicity of C. difficile.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### EXAMPLES

### Example 1

### Manufacture of Freeze Dried L. Reuteri Powder, with Loaded Microcompartments Containing Reuterin Activated During the Fermentation Step

Fermentation Media Composition

Dextrose mono hydrate 60 g/l

Yeast extract KAV 20 g/l

Peptone Type PS 20 g/l

Di ammonium hydrogen citrate 5 g/l

Sodium acetate (×3 H₂O) 4.7 g/l

Di potassium hydrogen phosphate 2 g/l

Tween 80 0.5 g/l

Silibione (anti foam) 0.14 g/l

Magnesium sulphate 0.10 g/l

Manganese sulphate 0.03 g/l

Zinc sulphate hepta hydrate 0.01 g/l

Water q.s.

Centrifuge Media

Peptone O-24 Orthana

Cryoprotectants

Lactose 33%

Gelatin hydrolysate 22%

Sodium glutamate 22%

Maltodextrin 11%

Ascorbic acid 11%

Production steps of freeze dried Lactobacillus reuteri powder

Twenty ml of the media is inoculated with 0.6 ml of freeze-dried Lactobacillus reuteri powder from a working cell bank vial. The fermentation is performed in a bottle at 37° C. for 18-20 hours without stirring or pH control.

Two 1-liter flasks of the media are inoculated with 9 ml cell slurry per liter. The fermentation is performed at 37° C. for 20-22 hours without stirring or pH control i.e. statical.

The two one-liter cell slurries from step no. 2 inoculates the 600-liter vessel. The fermentation is performed at 37° C. for 13 hours with stirring and pH control. At the start of the fermentation the pH is 6.5. The pH control starts when the pH drops below 5.4 using a 20% sodium hydroxide solution. The pH control is set to pH 5.5.

The fourth and final fermentation step is performed in a 15000-liter vessel with the inoculation from step no 3. The fermentation is performed at 37° C. for 9 to 12 hours with stirring and pH control. At the start of the fermentation the pH is 6.5. The pH control starts when the pH drops below 5.4 using a 20% sodium hydroxide solution. The pH control is set to pH 5.5. 99 liters of 87% glycerol is added in the final phase of the fermentation (giving the concentration 100 mM), just before the culture reaches the stationary phase. The fermentation is complete when the culture reaches the stationary phase, which can be seen by the reduction of the addition of the sodium hydroxide solution. Roughly 930 liters of the sodium hydroxide solution is added to the 10200 liters of media and 600 liters of inoculum during the fermentation.

The cell slurry from the final fermentation is separated at 10° C. twice in a continuous centrifuge from Alfa Laval. After the first centrifugation the volume of the cell slurry is reduced from roughly 11829 liters to 1200 liters. This volume is washed with 1200 liters of a peptone (Peptone 0-24, Orthana) solution in a 3000-liter vessel and is separated again before the mixing with the cryoprotectants. The washing step with peptone is performed to avoid any freezing-point reduction in the freeze-drying process.

After the second centrifugation the volume of the cell slurry is reduced to 495 liters. This volume is mixed with 156 kg of the cryoprotectant solution to reach roughly 650 liters of the cell slurry.

The cell slurry is pumped to a 1000-liter vessel. The vessel is then transported to the freeze-drying plant.

At the freeze-drying plant, exactly 2 liters of the cell slurry is poured on each plate in the freeze dryer. The maximum capacity of the freeze dryer is 600 liters and all excessive cell slurry volume is thrown away.

The cell slurry of Lactobacillus reuteri has a dry matter content of 18% and is freeze dried for four to five days.

During the freeze-drying process, the pressure in the process is between 0.176 mbar and 0.42 mbar. The vacuum pump is started when the pressure reaches 0.42 mbar. The PRT (pressurizing test) is used to determine when the process is complete. If the PRT or the increase of pressure is less than 0.02 mbar after 120 seconds, the process is stopped.

### Example 2

### Manufacture of Freeze Dried L. Reuteri Powder, with Loaded Microcompartments Containing Reuterin Primed and Activated During the Fermentation Step

Production process like in EXAMPLE 1 but primed with additional 200 mM 1,2-PD, vitamin C (4 g/l) and of vitamin B12 (1 μg/ml) in the growth media.

### Example 3

### Manufacture of Freeze Dried L. Reuteri Powder, with Loaded Microcompartments Containing Reuterin, Primed During the Fermentation Step and Activated for Reuterin Formation Before the Freeze-Drying Step

Production process like in EXAMPLE 1 but primed with additional 200 mM 1,2-PD, vitamin C (4g/l) and vitamin B12 (1 μg/ml) to the growth media. But without 100 mM glycerol added in the fermentation phase but instead added to the cell slurry before transported to the freeze-drying plant.

### Example 4

### Preparation of a Product in the Form of Gelatin Capsules Containing Freeze Dried L. Reuteri Powder, with Activated Reuterin-Production Machinery

A product is prepared from the following components:
Freeze-dried powder of L. reuteri, with activated reuterin-production machinery, manufactured by for example any of the manufacturing methods described above in Examples 1-3.
Gelatin capsules

The freeze-dried powder of L. reuteri is distributed into gelatin capsules (size 1, Coni-Snap, Capsugel).

### Example 5

### Preparation of a Product in the Form of Enteric Capsules Containing Freeze Dried L. Reuteri Powder, with Activated Reuterin-Production Machinery

A product is prepared from the following components:
Freeze-dried powder of L. reuteri, with activated reuterin-production machinery, manufactured by for example any of the manufacturing methods described above in Examples 1-3.
Enteric capsules

The freeze-dried powder of L. reuteri is distributed into VCAPS enteric capsules (size 1, Capsugel.

### Example 6

### L. Reuteri Strains Intrinsically Resistant to Antibiotics Used to Treat CDI

Strains used in this study are listed in Table 1. Routine culturing of Lactobacillus spp. in deMan, Rogosa, Sharpe medium (MRS; Difco, Franklin Lakes, N.Y.) and C. difficile strains in Brain Heart Infusion media (BHI; BD Biosciences, Franklin Lakes, NJ) was carried out at 37° C. in an anaerobic chamber (Anaerobe Systems, AS-580, Morgan Hill, Calif.) supplied with a mixture of 10% CO₂, 5% H₂, and 85% N₂ for 16-18 h.

Antibiotic Susceptibility Testing. Susceptibility of Lactobacillus spp. to antibiotics was determined using standard broth microdilution procedures (24). Briefly, bacteria (1×10⁶ CFU/mL) were inoculated into a 96-well plate containing serial dilutions of vancomycin (0.125-256 μg/mL), metronidazole (0.125-256 μg/mL), or fidaxomycin (0.016-32 μg/mL) in MRS. Plates were incubated anaerobically at 37° C. for 24 h. Optical density measurements (600 nm) were recorded using a Synergy™ H1 Hybrid Multi-Mode Microplate Reader (BioTek Instruments, Inc, Winooski, Vt.). Results were compared to a growth control (lactobacilli only), and the minimum inhibitory concentration (MIC) endpoint was the concentration of antibiotic where ≥90% reduction in growth was observed. Significance was determined using student's t test with equal variance.

Statistics

Results are presented as mean values±standard deviation (S.D.) or standard error of mean (S.E.M.) as indicated. Statistical significance was determined using t test, Mann-Whitney U, Kruskal-Wallis, ANOVA, AMOVA or ANOSIM with or without multiple testing correction; the precise test is specified in each section. Replicates (n) of 3 were assayed unless otherwise stated; p<0.05 was considered statistically significant.

Intrinsic resistance to multiple antibiotics is common in Lactobacillus spp., although the taxonomic complexity of the genus has made defining their antimicrobial susceptibilities difficult (45). To identify potentially useful probiotics in combatting recurrent CDI, we determined the minimum inhibitory concentrations (MICs) of specific human-derived strains of potential probiotics L. casei, L. gasseri, L. rhamnosus, and L. reuteri to vancomycin, metronidazole and fidaxomicin—drugs currently used to treat CDI (Table 1). A recent U.S.-based surveillance study of drug susceptibilities in C. difficile-associated diarrheal isolates showed the MIC₉₀ for vancomycin, metronidazole, and fidaxomicin to be 4, 2, and 0.5 μg/mL, respectively (46). L. casei LC-39 and L. rhamnosus LR-34, known to produce factors that modulate inflammation stimulated by C. difficile in vitro (47), demonstrated substantial resistance to vancomycin and metronidazole with MIC values >256 μg/mL; 64 to 128-fold greater than the vancomycin and metronidazole MIC₉₀ for C. difficile. However, these strains were susceptible to fidaxomicin at 2 μg/mL; only 4-fold greater than the fidaxomicin MIC₉₀ for C. difficile and much lower than estimated colonic concentrations of fidaxomicin in patients (48). L. gasseri LG-3, an isolate from human infant feces, was resistant to metronidazole (>256 μg/mL) and susceptible to vancomycin and fidaxomicin at 1 and 2 μg/mL, respectively. Human-derived L. reuteri strains DSM 17938 and ATCC PTA 6475 demonstrated the most consistent and robust resistance to all three drugs ranging from 64- to 128-fold greater than the corresponding MIC₉₀ for C. difficile. In all, L. reuteri strains exhibited the lowest susceptibility to antibiotics used to treat CDI potentiating their survival capabilities if given simultaneously with these drugs, and were screened further as potential probiotics for CDI prevention.

TABLE 1
Minimum inhibitory concentrations of Lactobacillus spp.
indicate resistance to antibiotics associated with increased
risk to CDI at clinically relevant concentrations.

| | | Vanco- | Metroni- | Fidaxo- |
|---|---|---|---|---|
| | | mycin | dazole | mycin |
| Strain | Source | (μg/mL) | (μg/mL) | (μg/mL) |
| C. difficile R20291 | CDI patient | 4 | 2 | 0.5 |
| | stool | | | |
| L. casei LC-39 | Infant feces | 256 | >256 | 2 |
| L. gasseri LG-3 | Infant feces | 1 | >256 | 2 |
| L. rhamnosus LR-34 | Infant feces | 256 | >256 | 2 |
| L. reuteri 17938 | Breastmilk | 256 | >256 | >32 |
| L. reuteri 6475 | Breastmilk | 256 | 128 | >32 |

### Example 7

### Strain-Specific, Reuterin-Dependent Antimicrobial Effects of L. Reuteri on C. Difficile Growth In Vitro

Strains used in this study are L. reuteri DSM 17938 (herein, sometimes called L. reuteri 17938), L. reuteri ATCC PTA 6475, C. difficile 820291, C. difficile 10463 and C. difficile 630. Routine culturing of Lactobacillus spp. in deMan, Rogosa, Sharpe medium (MRS; Difco, Franklin Lakes, N.Y.) and C. difficile strains in Brain Heart Infusion media (BHI; BD Biosciences, Franklin Lakes, N.J.) was carried out at 37° C. in an anaerobic chamber (Anaerobe Systems, AS-580, Morgan Hill, Calif.) supplied with a mixture of 10% CO₂, 5% H₂, and 85% N₂ for 16-18 h. Erythromycin (Erm, 10 μg/mL) or chloramphenicol (Cm, 10 μg/mL) was added when necessary for plasmid or chromosomal insertion maintenance.

Pathogen Inhibition Assay. C. difficile susceptibility to reuterin was measured using an agar spot test optimized to promote reuterin production by L. reuteri. Assays were performed as previously described (25) with minimal modifications. Briefly, overnight cultures of L. reuteri were spotted (2 μL) onto BHI supplemented with 20 mM glucose and developed by anaerobic incubation at 37° C. for 24 h. Overnight cultures of C. difficile strains were inoculated (10⁷-10⁸ cells/mL) in 7 mL soft agar (BHI broth, 2% glycerol, 0.7% technical agar), layered over the L. reuteri spots and incubated anaerobically at 37° C. for 24 hr. Clear zones of inhibition (≥1 mm) around each spot were scored. Significance was determined using student's t test with equal variance.

Statistics

Results are presented as mean values±standard deviation (S.D.) or standard error of mean (S.E.M.) as indicated. Statistical significance was determined using t test, Mann-Whitney U, Kruskal-Wallis, ANOVA, AMOVA or ANOSIM with or without multiple testing correction; the precise test is specified in each section. Replicates (n) of 3 were assayed unless otherwise stated; p<0.05 was considered statistically significant.

Many human-derived L. reuteri strains metabolize glycerol into the secondary three carbon aldehyde reuterin, with broad-spectrum antimicrobial activity towards enteric pathogens (49). Regulation of reuterin production is modulated by PocR, an AraC-like transcriptional regulator present in the 57-gene pdu-cbi-hem-cob cluster. Inactivation of the pocR gene inhibits both reuterin and vitamin B₁₂ synthesis (50). We previously demonstrated strain-dependent reuterin production by L. reuteri strains 17938 and 6475 showing that the former strain produces 3-fold more reuterin (25, 49), leading us to hypothesize that an L. reuteri strain with greater capacity for reuterin production would be more efficient at inhibiting C. difficile growth in vitro.

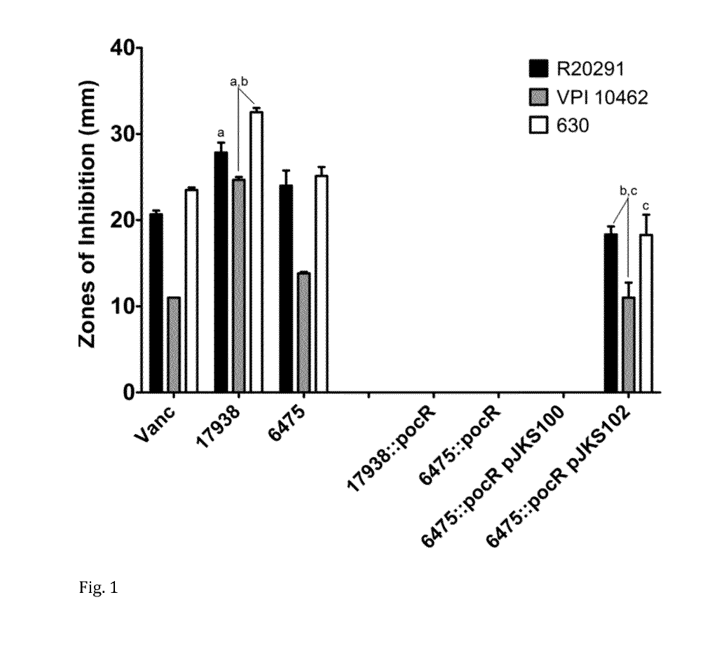

The strain-dependent effects of L. reuteri on C. difficile growth were evaluated using an agar spot overlay assay optimized for reuterin production (25). We tested wild-type L. reuteri 17938 and 6475 strains alongside isogenic pocR mutants, 17938::pocR and 6475::pocR incapable of producing reuterin (50), for activity against C. difficile strains VPI 10463, 820291, and 630 (FIG. 1). A disc of vancomycin (5 μg) was included as a positive control for growth inhibition of C. difficile. All C. difficile strains were susceptible to wild type L. reuteri strains 17938 and 6475. No growth inhibition was seen by the pocR mutants, and inhibition of C. difficile was restored when strain 6475::pocR was complemented with the wild-type pocR gene (6475::pocR pJKS102) but not empty vector (6475::pocR pJKS100) (FIG. 1). Differential inhibitory activity was observed between L. reuteri strains, with 17938 exhibiting the greatest growth inhibition of C. difficile VPI 10463 and 820291 as compared to 6475 and vancomycin control (p<0.05; FIG. 1). Assays conducted in the absence of glycerol did not result in C. difficile growth inhibition (data not shown). Taken together, these data show that in the presence of glycerol human-derived L. reuteri strains have antimicrobial activity against C. difficile in vitro and reuterin production is required for this phenotype.

### Example 8

### Glycerol Fermentation by L. Reuteri DSM 17938 Prevents C. Difficile Invasion in Antibiotic-Treated Human Fecal Mini-Bioreactor Arrays

Mini-bioreactor array (MBRA) preparation, operation and sample collection. Effects of reuterin on C. difficile growth in a human fecal microbial community were tested in MBRAs. Replicate MBRAs (twelve independent 15-mL reactors) and bioreactor medium (BRM2) were prepared as previously described (26). Reactors were inoculated with an anaerobic preparation of a 25% fecal slurry from an anonymous healthy donor (5% w/v final concentration) and were operated in an anaerobic chamber with 5% H2, 5% CO2, 90% N2 atmosphere. After 16 h of outgrowth in batch culture mode, continuous-flow cultivation was initiated at a flow rate of 0.94 ml/h (16 h retention time). After 36 h of flow, reactor communities were treated twice daily with clindamycin (500 mg/L final concentration) for 4.5 days. Reactors were transitioned to fresh medium on day 4 of clindamycin treatment, with 6 reactors receiving standard BRM2 medium and 6 reactors receiving BRM2 supplemented with glycerol (10% v/v final concentration). Reactors continued on respective medium throughout the duration of the experiment. Thirteen hours after cessation of antibiotic treatment, six reactors were treated with L. reuteri DSM 17938 (3 reactors in BMR2 and 3 reactors in BRM2+glycerol) prepared as described below. L. reuteri treatment continued twice daily for 3 days. After the first day of L. reuteri treatment, all 12 reactors were challenged with ˜1×106 vegetative C. difficile CD2015 (clinical ribotype 027) that had been propagated in BRM2 prior to inoculation (27).

Samples (1 ml) were collected from each MBRA as described (27) prior to the initiation of continuous flow and then daily through the duration of the experiment. Samples were centrifuged at 20,000×g for 1 min, then supernatants and pellets were separated and stored at −80° C. Microbial community and metabolite analysis, and quantification of C. difficile levels by quantitative PCR (qPCR) were performed as described below.

L. reuteri cultivation and dosing for MBRAs. L. reuteri colonies growing anaerobically at 37° C. on MRS agar (≤2 days) were inoculated into 10 ml of MRS broth and incubated anaerobically at 37° C. for 8-12 h. Two 3-ml aliquots of culture were removed, concentrated by centrifugation for 5 min at 1,753×g, and washed in BRM2 or BRM2+10% glycerol. Bacterial cell pellets were resuspended in 1 ml BRM2 or BRM2+10% glycerol and incubated at 37° C. anaerobically for 15 min prior to dosing 300 μl into reactors. Aliquots of the L. reuteri inocula were serially diluted, plated on MRS agar, and incubated anaerobically overnight at 37° C. to determine colony forming units (CFU)/ml of inoculum. CFU/ml were 5.2±4.2×109 CFU/ml and 3.4±3.9×109 CFU/ml for BRM2 and BRM2+glycerol incubated cultures, respectively.

Microbial DNA Extraction. Stool, intestinal content and MBRA samples were extracted using methods described previously with modifications (30, 31). Briefly, samples were suspended in pre-heated lysis buffer (65° C.) and subjected to two cycles of homogenization using the BeadBlaster Tissue Homogenizer (Benchmark Scientific, Melrose, MA) for 20s at 6.00 M/s. Remaining intact cells and debris were pelleted by centrifugation for 5 min at 5,000×g and supernatant collected. An additional 300 μl of pre-heated lysis buffer was added to the pellet, homogenization and centrifugation repeated, and supernatants pooled. A mixture of ammonium acetate (2M final concentration) and pooled supernatant was incubated on ice for 10 min, and then centrifuged at 4° C. for 10 min at 14,000×g. The supernatant was collected, mixed with equal volume of isopropanol, and incubated overnight at −20° C. Precipitation, washing, and removal of RNA and protein was performed as described by Yu (30). DNA was purified using the Zymo Research DNA clean and concentrator 25 kit (Irvine, CA) according to the manufacturer's instructions and stored at −20° C. until further analysis by 16S rRNA gene sequencing or qPCR.

Quantification of C. difficile by qPCR. Real time qPCR was used to determine the amount of C. difficile 16S rRNA gene relative to total bacterial 16S rRNA gene in samples from MBRAs, intestinal contents or stool. Template DNA was extracted as described above and 10 ng used in reactions with SYBR® Green PCR Master Mix (Applied Biosystems™, Waltham, Mass.) according to the manufacturer's instructions. Bacterial 16S primers were used to assess total bacterial 16S rRNA gene (forward 5′-GCA GGC CTA ACA CAT GCA AGT C (SEQ ID NO:1) and reverse 5′-CTG CTG CCT CCC GTA GGA GT (SEQ ID NO:2)) (32) or C. difficile 16S rRNA gene (forward 5′-TTG AGC GAT TTA CTT CGG TAA AGA (SEQ ID NO:3) and reverse 5′-CCA TCC TGT ACT GGC TCA CCT (SEQ ID NO:4)) (33). Real time quantification was carried out on a MJ Research PTC-200 thermocycler (Bio-Rad, Hercules, Calif.) with the following cycling conditions: initial 95° C. for 5 min, then 41 cycles of 95° C. for 20 sec, 60° C. for 1 min, and 84° C. for 1 sec, followed by a melting curve. Relative quantification of C. difficile 16S rRNA gene was calculated with the 2^{-ΔΔC}^{_{T}} method (34). Significance in relative C. difficile abundance between groups was determined using unpaired, two-tailed t tests with unequal variance run over 7 time points using pairwise comparisons of three treatments versus control.
16S rRNA Gene Sequencing. Microbial DNA extracted as outlined above was sequenced as previously described by the Human Microbiome Project (35). Sequencing of the V3V5 region of the 16S rRNA gene was accomplished on the 454 GSFLX platform (Roche) with the forward primer 357F (CCTACGGGAGGCAGCAG (SEQ ID NO:5)) and adapter-tagged reverse primer 534R (CCGTCAATTCMTTTRAGT (SEQ ID NO:6)).

### Statistics

Results are presented as mean values±standard deviation (S.D.) or standard error of mean (S.E.M.) as indicated. Statistical significance was determined using t test, Mann-Whitney U, Kruskal-Wallis, ANOVA, AMOVA or ANOSIM with or without multiple testing correction; the precise test is specified in each section. Replicates (n) of 3 were assayed unless otherwise stated; p<0.05 was considered statistically significant.

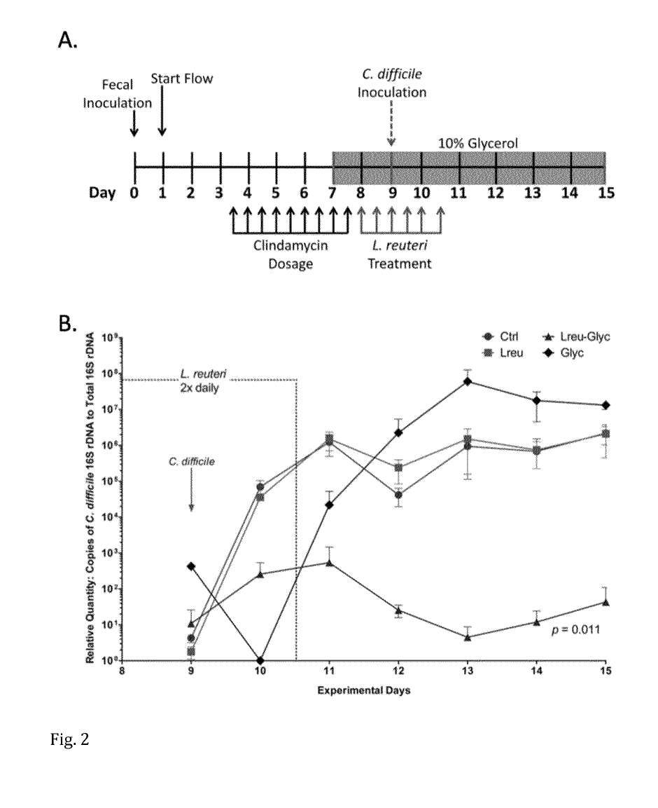

Fecal mini-bioreactor arrays (MBRAs) were established by Robinson, et al (27) to model C. difficile invasion of antibiotic-disrupted microbial communities. In this model, human fecal samples from C. difficile-negative adult donors are inoculated into multiple continuous-flow MBRAs. After allowing microbial communities to establish in anaerobic culture, communities were disrupted by antibiotic treatment and infected with C. difficile (FIG. 2A). The antibiotic clindamycin predisposes intestinal microbial communities to C. difficile invasion (54) and is used to disrupt the fecal MBRAs; those not treated with clindamycin are resistant to C. difficile colonization (27). We utilized this model to determine if production of reuterin by L. reuteri could prevent C. difficile invasion of an antibiotic treated microbial community in MBRAs. Additionally, high-throughput sequencing of 16S rRNA genes were employed to determine how the microbial communities in the C. difficile-infection MBRA model compared to CDI patient microbial communities.

We modified the MBRA model to study the effects of L. reuteri and reuterin production on C. difficile invasion of antibiotic-treated microbial communities (FIG. 2A). Specifically, we tested whether the addition of glycerol alone (Glyc), L. reuteri alone (Lreu) or L. reuteri and glycerol together (Lreu-Gly) altered C. difficile invasion dynamics compared to untreated reactors (Ctrl). We monitored the abundance of C. difficile relative to total bacterial load in MBRAs using qPCR with primers specific to the C. difficile 16S rRNA gene and universal 16S rRNA gene on days 9-15 (FIG. 2B; Day 9 was collected prior to C. difficile addition). We found that C. difficile levels in Lreu-Glyc MBRAs decreased significantly (˜10⁵-fold lower than untreated reactors, p=0.011). L. reuteri treatment alone did not significantly alter C. difficile levels compared to controls whereas glycerol alone resulted in ˜10-fold greater concentrations of C. difficile, although this increase was not statistically significant.

### Example 9

### C. Difficile Growth and Toxicity is Diminished by L. Reuteri In Vivo

Mouse model of recurrent CDI. The effect of supplementation with L. reuteri and glycerol on CDI was tested in a mouse model of recurrent disease. The UTMB Institutional Animal Care and Use Committee approved all experiments. Eight-week old C57BL/6 female mice were purchased from Jackson Laboratory (Sacramento, Calif.). Mice were given an antibiotic cocktail (43) administered in their drinking water three days prior to infection, and then switched to regular drinking water. One day prior to infection, all mice received an i.p. injection of clindamycin (10 mg/kg) and began daily oral gavages of 5×10⁹ CFU of L. reuteri (in logarithmic growth phase) suspended in fresh MRS media containing 10% glycerol. Prior to dosing the bacterial cell pellets were resuspended in 1 ml MRS and 10% glycerol and incubated at 37° C. anaerobically for 30 min. Control animals were given fresh MRS media with 10% glycerol without bacteria. All mice were infected by oral gavage with 10³ CFU spores of C. difficile VPI 10463 and treated orally with vancomycin (40 mg/kg) for 5 days. C. difficile burden was assessed from feces homogenized in deoxygenated PBS and cultured on pre-reduced selective taurocholate-cycloserine-cefoxitin BHI agar plates under anaerobic conditions at 37° C. (44). Statistical significance was determined using an unpaired, two-tailed t test with unequal variances.
Mice were assessed for morbidity and/or mortality as well as clinical symptoms for the duration of the experiment. At the end of the experiment, organs were collected for histopathological analysis. Severity of damage in the cecum and colon was assessed and scored on a scale of 0-5 as follows: (0) Normal and no damage; (1) mild, focal infiltration, no epithelial cell loss; (2) moderate infiltration, no epithelial cell loss; (3) extensive infiltration, no epithelial cell los; (4) moderate infiltration with epithelial cell loss; and (5) extensive infiltration with epithelial cell loss, congestion, and blood. Scores were assessed by two blinded observers and then averaged. Statistical significance was determined using an unpaired, two-tailed t test with Welch's multiple testing correction.
Cytotoxicity of C. difficile toxin in stool. Chinese hamster ovary KI cells (CHO, ATCC CCL-61) were cultured in F12 medium supplemented with 10% FCS and grown to confluency prior to seeding in 96-well microtiter plates. The plates were incubated for 24 h at 37° C. in 5% CO₂ before experimental use. Fecal pellets were homogenized in PBS (10 mg/mL), centrifuged at 9,000×g for 5 min, and the supernatant collected and filter sterilized. Supernatants were serially diluted 10-fold in F12 medium, applied to plate wells and incubated for 24 h at 37° C. in 5% CO₂. Each plate included control wells inoculated with purified C. difficile toxin B (List Laboratories, Campbell, Calif.) or antitoxin (TechLab, Blacksburg, Va.). Statistical significance was determined using unpaired, two-tailed t test with unequal variances.

### Statistics

Results are presented as mean values±standard deviation (S.D.) or standard error of mean (S.E.M.) as indicated. Statistical significance was determined using t test, Mann-Whitney U, Kruskal-Wallis, ANOVA, AMOVA or ANOSIM with or without multiple testing correction; the precise test is specified in each section. Replicates (n) of 3 were assayed unless otherwise stated; p<0.05 was considered statistically significant.

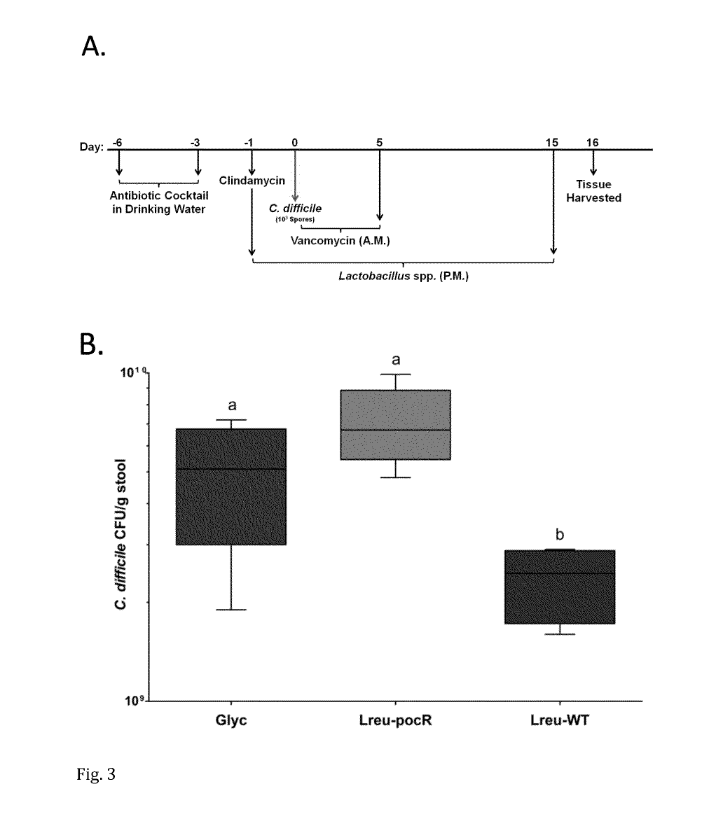

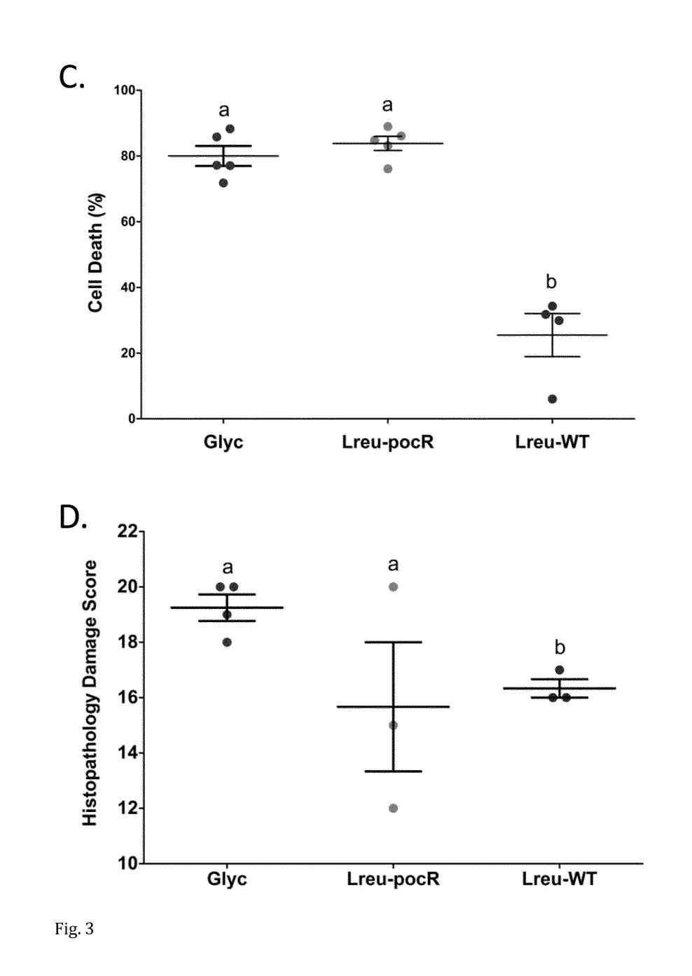

To determine the efficacy of the co-delivery of L. reuteri and glycerol on disease severity in a mouse model of recurrent CDI infection (FIG. 3A), antibiotic-treated mice were gavaged daily following clindamycin administration on day −1 with 10% glycerol either alone (Glyc), with L. reuteri 17938 wild-type (Lreu-WT; reuterin producer), or with L. reuteri 17938::pocR (Lreu-pocR; non-reuterin producer). Animals were infected with C. difficile VPI 10463 spores and disease severity was evaluated with daily health scoring, C. difficile burden and TcdA toxicity in stool, and by blinded histopathologic analysis of intestinal tissue sections. Stool samples (day 2) from Lreu-WT animals showed a modest although significant reduction in C. difficile concentrations (p<0.05) along with significantly less TcdA-mediated cytotoxicity (p<0.0001) than stool from Glyc or Lreu-pocR animals (FIG. 3B & C). No significant differences were seen in C. difficile burden or toxicity between Glyc and Lreu-pocR animals (FIG. 3B & C). Histopathology scoring indicated less cellular damage and inflammation in cecal tissue of Lreu-WT animals compared to Glyc controls (FIG. 3D). Additionally, average histopathology was less in Lreu-pocR animals, nearing that seen in Lreu-WT.

### Example 10

### Inhibition of C. Difficile CD2015 by Preloaded L. Reuteri 17938 is Both gdh and Concentration Dependent

Bacterial Strains and Culture Conditions. Routine culturing of Lactobacillus reuteri strains 17938 (wild-type) and 17938::gdh (reuterin mutant) in deMan, Rogosa, Sharpe medium (MRS; Difco, Franklin Lakes, N.Y.) and C. difficile CD2015 in Brain Heart Infusion medium with 2% D-glucose (w/v) (BHI; BD Biosciences, Franklin Lakes, N.J.) was carried out at 37° C. in an anaerobic chamber (Anaerobe Systems, AS-580, Morgan Hill, Calif.) supplied with a mixture of 10% CO₂, 5% H₂, and 85% N₂ for 16-18 h. Erythromycin (Erm, 10 μg/mL) was added to MRS when routinely culturing L. reuteri 17938::gdh for chromosomal insertion maintenance.

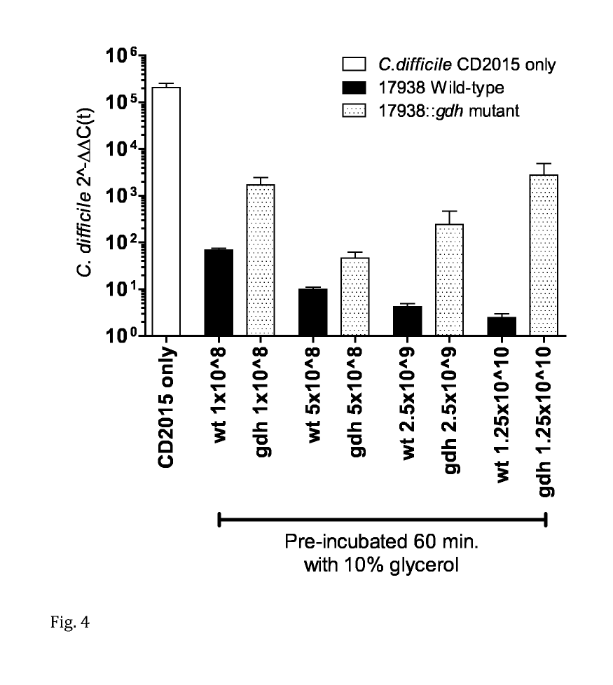

Pathogen Inhibition Assay. C. difficile CD2015 susceptibility to loaded L. reuteri 17938 or 17938::gdh was measured in an in vitro co-culture assay. Briefly, L. reuteri colonies growing anaerobically at 37° C. on MRS agar (≤2 days) were inoculated into 10 ml of MRS broth and incubated anaerobically at 37° C. for 12 h. Cells were concentrated by centrifugation for 5 min at 2,000×g, and washed in 50 mM sodium phosphate buffer. Bacterial cell pellets were resuspended in 10% glycerol and incubated at 37° C. anaerobically for 60 min. Cells were collected by centrifugation and washed in 50 mM sodium phosphate buffer prior to dosing at designated concentrations into BHI medium with 2% D-glucose for co-culture reactions. Each co-culture reaction was subsequently inoculated with overnight cultures of C. difficile CD2015 (10⁴ cells/mL) and incubated anaerobically at 37° C. for 24 hr. Samples were taken at 24 h and C. difficile CD2015 bacteria were quantified by qPCR as described below.
Microbial DNA Extraction. Co-culture samples were extracted using methods described previously with modifications (30, 31). Briefly, samples were suspended in pre-heated lysis buffer (70° C.) and subjected to two cycles of homogenization using the BeadBlaster Tissue Homogenizer (Benchmark Scientific, Melrose, Mass.) for 20s at 6.00 M/s. Remaining intact cells and debris were pelleted by centrifugation for 5 min at 13,000×g and supernatant collected. A mixture of ammonium acetate (2M final concentration) and pooled supernatant was incubated on ice for 5 min, and then centrifuged at 4° C. for 10 min at 13,000×g. The supernatant was collected, mixed with equal volume of isopropanol, and incubated overnight at −20° C. Precipitation, washing, and removal of RNA and protein was performed as described by Yu (30). DNA was purified using the Zymo Research DNA clean and concentrator 25 kit (Irvine, Calif.) according to the manufacturer's instructions and stored at −20° C. until further analysis by 16S rRNA gene sequencing or qPCR.
Quantitation of C. difficile by qPCR. Real time qPCR was used to determine the amount of C. difficile CD2015 16S rRNA gene relative to total bacterial 16S rRNA gene in samples from MBRAs or intestinal contents as previously reported (34, 77 and 78). Template DNA was extracted as described above and 5 ng used in reactions with Kap SYBR®FAST qPCR Master Mix (Kapa Biosystems™, Wilmington, Mass.) according to the manufacturer's instructions. Bacterial 16S primers were used to assess total bacterial 16S rRNA gene (forward 5′-GCA GGC CTA ACA CAT GCA AGT C and reverse 5′-CTG CTG CCT CCC GTA GGA GT) (6) or C. difficile 16S rRNA gene (forward 5′-TTG AGC GAT TTA CTT CGG TAA AGA and reverse 5′-CCA TCC TGT ACT GGC TCA CCT) content (7). Real time quantification was carried out on a Roche LightCycler thermocycler (Roche Molecular Systems, Inc., Pleasanton, Calif.) with the following cycling conditions: initial 95° C. for 3 min, then 41 cycles of 95° C. for 10 sec, 60° C. for 10 sec, and 72° C. for 10 sec, followed by a melting curve. Relative quantification of C. difficile 16S rRNA gene was calculated with the 2^{-ΔΔC}^{_{T}} method (34, 77 and 78). The results are shown in FIG. 4.

### REFERENCES

1. (CDC) CfDCaP (ed). 2013. Antibiotic Resistance Threats in the United States, 2013. U.S. Department of Health and Human Services, Centers for Disease Control and Prevention,

2. Lessa F C, Mu Y, Bamberg W M, Beldays Z G, Dumyati G K, Dunn J R, Farley M M, Holzbauer S M, Meek J I, Phipps E C, Wilson L E, Winston L G, Cohen J A, Limbago B M, Fridkin S K, Gerding D N, McDonald L C. 2015. Burden of Clostridium difficile infection in the United States. N Engl J Med 372:825-834.

3. Desai K, Gupta S B, Dubberke E R, Prabhu V S, Browne C, Mast T C. 2016. Epidemiological and economic burden of Clostridium difficile in the United States: estimates from a modeling approach. BMC Infect Dis 16:303.

4. Garey K W, Sethi S, Yadav Y, DuPont H L. 2008. Meta-analysis to assess risk factors for recurrent Clostridium difficile infection. J Hosp Infect 70:298-304.

5. Kelly C P. 2012. Can we identify patients at high risk of recurrent Clostridium difficile infection? Clin Microbiol Infect 18 Suppl 6:21-27.

6. McFarland L V, Elmer G W, Surawicz C M. 2002. Breaking the cycle: treatment strategies for 163 cases of recurrent Clostridium difficile disease. Am J Gastroenterol 97:1769-1775.

7. Ofosu A. 2016. Clostridium difficile infection: a review of current and emerging therapies. Ann Gastroentero129:147-154.

8. Anonymous. Guidance for industry. Enforcement policy regarding investigational new drug requirements for use of fecal microbiota for transplantation to treat Clostridium difficile infection not responsive to standard therapies.

9. Bacteria TFfCA-R (ed). 2015. National Action Plan for Combating Antibiotic-Resistant Bacteria. The White House, Washington, D.C.

10. McFarland L V. 2016. Therapies on the horizon for Clostridium difficile infections. Expert Opin Investig Drugs 25:541-555.

11. Bakken J S. 2014. Staggered and tapered antibiotic withdrawal with administration of kefir for recurrent Clostridium difficile infection. Clin Infect Dis 59:858-861.

12. Maziade P J, Andriessen J A, Pereira P, Currie B, Goldstein E J. 2013. Impact of adding prophylactic probiotics to a bundle of standard preventative measures for Clostridium difficile infections: enhanced and sustained decrease in the incidence and severity of infection at a community hospital. Curr Med Res Opin 29:1341-1347.

13. Maziade P J, Pereira P, Goldstein E J. 2015. A Decade of Experience in Primary Prevention of Clostridium difficile Infection at a Community Hospital Using the Probiotic Combination Lactobacillus acidophilus CL1285, Lactobacillus casei LBC80R, and Lactobacillus rhamnosus CLR2 (Bio-K+). Clin Infect Dis 60 Suppl 2:S144-147.

14. Spinler J K, Ross C L, Savidge T C. 2016. Probiotics as adjunctive therapy for preventing Clostridium difficile infection—What are we waiting for? Anaerobe.

15. Cohen S H, Gerding D N, Johnson S, Kelly C P, Loo V G, McDonald L C, Pepin J, Wilcox M H. 2010. Clinical practice guidelines for Clostridium difficile infection in adults: 2010 update by the society for healthcare epidemiology of America (SHEA) and the infectious diseases society of America (IDSA). Infect Control Hosp Epidemiol 31:431-455.

16. Pamer E G. 2016. Resurrecting the intestinal microbiota to combat antibiotic-resistant pathogens. Science 352:535-538.

17. Jose N M B, C. R.; Hussain, M. A. 2015. Implications of Antibiotic Resistance in Probiotics. Food Reviews International 31:52-62.

18. Vollenweider S, Grassi G, Konig I, Puhan Z. 2003. Purification and structural characterization of 3-hydroxypropionaldehyde and its derivatives. J Agric Food Chem 51:3287-3293.

19. Casas I A, Dobrogosz W J. 2000. Validation of the probiotic concept: Lactobacillus reuteri confers broad-spectrum protection against disease in humans and animals. Microb Ecol Health Dis 12:247-285.

20. Morita H, Toh H, Fukuda S, Horikawa H, Oshima K, Suzuki T, Murakami M, Hisamatsu S, Kato Y, Takizawa T, Fukuoka H, Yoshimura T, Itoh K, O'Sullivan D J, McKay L L, Ohno H, Kikuchi J, Masaoka T, Hattori M. 2008. Comparative genome analysis of Lactobacillus reuteri and Lactobacillus fermentum reveal a genomic island for reuterin and cobalamin production. DNA Res 15:151-161.

21. Santos F, Vera J L, van der Heijden R, Valdez G, de Vos W M, Sesma F, Hugenholtz J. 2008. The complete coenzyme B12 biosynthesis gene cluster of Lactobacillus reuteri CRL1098. Microbiol 154:81-93.

22. Sriramulu D D, Liang M, Hernandez-Romero D, Raux-Deery E, Lunsdorf H, Parsons J B, Warren M J, Prentice M B. 2008. Lactobacillus reuteri DSM 20016 produces cobalamin-dependent diol dehydratase in metabolosomes and metabolizes 1,2-propanediol by disproportionation. J Bacteriol 190:4559-4567.

23. Jacobsen C N, Rosenfeldt Nielsen V, Hayford A E, Moller P L, Michaelsen K F, Paerregaard A, Sandstrom B, Tvede M, Jakobsen M. 1999. Screening of probiotic activities of forty-seven strains of Lactobacillus spp. by in vitro techniques and evaluation of the colonization ability of five selected strains in humans. Appl Environ Microbiol 65:4949-4956.

24. NCCLS (ed). 2004. Methods for Antimicrobial Susceptibility Testing of Anaerobic Bacteria; Approved Standard-Sixth Edition. NCCLS, Wayne, Pa.

25. Spinler J K, Taweechotipatr M, Rognerud C L, Ou C N, Tumwasorn S, Versalovic J. 2008. Human-derived probiotic Lactobacillus reuteri demonstrate antimicrobial activities targeting diverse enteric bacterial pathogens. Anaerobe 14:166-171.

26. Auchtung J M, Robinson C D, Britton R A. 2015. Cultivation of stable, reproducible microbial communities from different fecal donors using minibioreactor arrays (MBRAs). Microbiome 3:42.

27. Robinson C D, Auchtung J M, Collins J, Britton R A. 2014. Epidemic Clostridium difficile strains demonstrate increased competitive fitness compared to nonepidemic isolates. Infect Immun 82:2815-2825.

28. Koenigsknecht M J, Theriot C M, Bergin I L, Schumacher C A, Schloss P D, Young V B. 2015. Dynamics and establishment of Clostridium difficile infection in the murine gastrointestinal tract. Infect Immun 83:934-941.

29. Buffie C G, Bucci V, Stein R R, McKenney P T, Ling L, Gobourne A, No D, Liu H, Kinnebrew M, Viale A, Littmann E, van den Brink M R, Jenq R R, Taur Y, Sander C, Cross J R, Toussaint N C, Xavier J B, Pamer E G. 2015. Precision microbiome reconstitution restores bile acid mediated resistance to Clostridium difficile. Nature 517:205-208.

30. Yu Z, Morrison M. 2004. Improved extraction of PCR-quality community DNA from digesta and fecal samples. BioTechniques 36:808-812.

31. Salonen A, Nikkila J, Jalanka-Tuovinen J, Immonen O, Rajilic-Stojanovic M, Kekkonen R A, Palva A, de Vos W M. 2010. Comparative analysis of fecal DNA extraction methods with phylogenetic microarray: effective recovery of bacterial and archaeal DNA using mechanical cell lysis. Journal of microbiological methods 81:127-134.

32. Castillo M, Martin-Orue S M, Manzanilla E G, Badiola I, Martin M, Gasa J. 2006. Quantification of total bacteria, enterobacteria and lactobacilli populations in pig digesta by real-time PCR. Vet Microbio1114:165-170.

33. Rinttila T, Kassinen A, Malinen E, Krogius L, Palva A. 2004. Development of an extensive set of 16S rDNA-targeted primers for quantification of pathogenic and indigenous bacteria in faecal samples by real-time PCR. J Appl Microbiol 97:1166-1177.

34. Livak K J, Schmittgen T D. 2001. Analysis of relative gene expression data using real-time quantitative PCR and the 2(-Delta Delta C(T)) Method. Methods 25:402-408.

35. Aagaard K, Petrosino J, Keitel W, Watson M, Katancik J, Garcia N, Patel S, Cutting M, Madden T, Hamilton H, Harris E, Gevers D, Simone G, McInnes P, Versalovic J. 2013. The Human Microbiome Project strategy for comprehensive sampling of the human microbiome and why it matters. FASEB J 27:1012-1022.

36. Edgar R C. 2013. UPARSE: highly accurate OTU sequences from microbial amplicon reads. Nat Methods 10:996-998.

37. Caporaso J G, Kuczynski J, Stombaugh J, Bittinger K, Bushman F D, Costello E K, Fierer N, Peña A G, Goodrich J K, Gordon J I, Huttley G A, Kelley S T, Knights D, Koenig J E, Ley R E, Lozupone C A, McDonald D, Muegge B D, Pirrung M, Reeder J, Sevinsky J R, Turnbaugh P J, Walters W A, Widmann J, Yatsunenko T, Zaneveld J, Knight R. 2010. QIIME allows analysis of high-throughput community sequencing data. Nat Methods 7:335-336.

38. Montrose D C, Zhou X K, Kopelovich L, Yantiss R K, Karoly E D, Subbaramaiah K, Dannenberg A J. 2012. Metabolic profiling, a noninvasive approach for the detection of experimental colorectal neoplasia. Cancer Prey Res 5:1358-1367.

39. Chumpitazi B P, Hollister E B, Oezguen N, Tsai C M, McMeans A R, Luna R A, Savidge T C, Versalovic J, Shulman R J. 2014. Gut microbiota influences low fermentable substrate diet efficacy in children with irritable bowel syndrome. Gut Microbes 5:165-175.

40. Beerenwinkel N, Antal T, Dingli D, Traulsen A, Kinzler K W, Velculescu V E, Vogelstein B, Nowak M A. 2007. Genetic progression and the waiting time to cancer. PLoS Comput Biol 3:e225.

41. Reitman Z J, Jin G, Karoly E D, Spasojevic I, Yang J, Kinzler K W, He Y, Bigner D D, Vogelstein B, Yan H. 2011. Profiling the effects of isocitrate dehydrogenase 1 and 2 mutations on the cellular metabolome. Proc Natl Acad Sci USA 108:3270-3275.

42. Evans A M, DeHaven C D, Barrett T, Mitchell M, Milgram E. 2009. Integrated, nontargeted ultrahigh performance liquid chromatography/electrospray ionization tandem mass spectrometry platform for the identification and relative quantification of the small-molecule complement of biological systems. Analytical chemistry 81:6656-6667.

43. Chen X, Katchar K, Goldsmith J D, Nanthakumar N, Cheknis A, Gerding D N, Kelly C P. 2008. A mouse model of Clostridium difficile-associated disease. Gastroenterol 135:1984-1992.

44. Jarchum I, Liu M, Lipuma L, Pamer E G. 2011. Toll-like receptor 5 stimulation protects mice from acute Clostridium difficile colitis. Infect Immun 79:1498-1503.

45. Goldstein E J, Tyrrell K L, Citron D M. 2015. Lactobacillus species: taxonomic complexity and controversial susceptibilities. Clin Infect Dis 60 Suppl 2:S98-107.

46. Snydman D R, McDermott L A, Jacobus N V, Thorpe C, Stone S, Jenkins S G, Goldstein E J, Patel R, Forbes B A, Mirrett S, Johnson S, Gerding D N. 2015. U.S.-Based National Sentinel Surveillance Study for the Epidemiology of Clostridium difficile-Associated Diarrheal Isolates and Their Susceptibility to Fidaxomicin. Antimicrob Agents Chemother 59:6437-6443.

47. Boonma P, Spinier J K, Venable S F, Versalovic J, Tumwasorn S. 2014. Lactobacillus rhamnosus L34 and Lactobacillus casei L39 suppress Clostridium difficile-induced IL-8 production by colonic epithelial cells. BMC Microbiol 14:177.

48. Sears P, Crook D W, Louie T J, Miller M A, Weiss K. 2012. Fidaxomicin attains high fecal concentrations with minimal plasma concentrations following oral administration in patients with Clostridium difficile infection. Clin Infect Dis 55 Suppl 2:S116-120.

49. Spinier J K, Sontakke A, Hollister E B, Venable S F, Oh PL, Balderas M A, Saulnier D M, Mistretta T A, Devaraj S, Walter J, Versalovic J, Highlander S K. 2014. From prediction to function using evolutionary genomics: human-specific ecotypes of Lactobacillus reuteri have diverse probiotic functions. Genome Biol Evol 6:1772-1789.

50. Santos F, Spinier J K, Saulnier D M, Molenaar D, Teusink B, de Vos W M, Versalovic J, Hugenholtz J. 2011. Functional identification in Lactobacillus reuteri of a PocR-like transcription factor regulating glycerol utilization and vitamin B12 synthesis. Microb Cell Fact 10:55.

51. Sung H W, Chen C N, Liang H F, Hong M H. 2003. A natural compound (reuterin) produced by Lactobacillus reuteri for biological-tissue fixation. Biomaterials 24:1335-1347.

52. Cleusix V, Lacroix C, Vollenweider S, Duboux M, Le Blay G. 2007. Inhibitory activity spectrum of reuterin produced by Lactobacillus reuteri against intestinal bacteria. BMC microbiology 7:101.

53. Schaefer L, Auchtung T A, Hermans K E, Whitehead D, Borhan B, Britton R A. 2010. The antimicrobial compound reuterin (3-hydroxypropionaldehyde) induces oxidative stress via interaction with thiol groups. Microbio1156:1589-1599.

54. Bartlett J G. 1981. Antimicrobial agents implicated in Clostridium difficile toxin-associated diarrhea of colitis. Johns Hopkins Med J Suppl 149:6-9.

55. Schubert A M, Rogers M A, Ring C, Mogle J, Petrosino J P, Young V B, Aronoff D M, Schloss P D. 2014. Microbiome data distinguish patients with Clostridium difficile infection and non-C. difficile-associated diarrhea from healthy controls. mBio 5:e01021-01014.

56. Arskold E, Lohmeier-Vogel E, Cao R, Roos S, Radstrom P, van Niel E W. 2008. Phosphoketolase pathway dominates in Lactobacillus reuteri ATCC 55730 containing dual pathways for glycolysis. Journal of bacteriology 190:206-212.

57. Biebl H, Menzel K, Zeng A P, Deckwer W D. 1999. Microbial production of 1,3-propanediol. Appl Microbiol Biotechnol 52:289-297.

58. Dinleyici E C, Dalgic N, Guven S, Metin O, Yasa O, Kurugol Z, Turel O, Tanir G, Yazar A S, Arica V, Sancar M, Karbuz A, Eren M, Ozen M, Kara A, Vandenplas Y. 2015. Lactobacillus reuteri DSM 17938 shortens acute infectious diarrhea in a pediatric outpatient setting. J Pediatr (Rio J) 91:392-396.

59. Dinleyici E C, Vandenplas Y. 2014. Lactobacillus reuteri DSM 17938 effectively reduces the duration of acute diarrhoea in hospitalised children. Acta Paediatr 103:e300-305.

60. Shornikova A V, Casas I A, Isolauri E, Mykkanen H, Vesikari T. 1997. Lactobacillus reuteri as a therapeutic agent in acute diarrhea in young children. J Pediatr Gastroenterol Nutr 24:399-404.

61. Cimperman L, Bayless G, Best K, Diligente A, Mordarski B, Oster M, Smith M, Vatakis F, Wiese D, Steiber A, Katz J. 2011. A randomized, double-blind, placebo-controlled pilot study of Lactobacillus reuteri ATCC 55730 for the prevention of antibiotic-associated diarrhea in hospitalized adults. J Clin Gastroenterol 45:785-789.

62. Tubelius P, Stan V, Zachrisson A. 2005. Increasing work-place healthiness with the probiotic Lactobacillus reuteri: a randomised, double-blind placebo-controlled study. Environ Health 4:25.

63. Cleusix V, Lacroix C, Vollenweider S, Le Blay G. 2008. Glycerol induces reuterin production and decreases Escherichia coli population in an in vitro model of colonic fermentation with immobilized human feces. FEMS microbiology ecology 63:56-64.

64. Martz S L, Guzman-Rodriguez M, He S M, Noordhof C, Hurlbut D J, Gloor G B, Carlucci C, Weese S, Allen-Vercoe E, Sun J, Claud E C, Petrof E O. 2016. A human gut ecosystem protects against C. difficile disease by targeting TcdA. Journal of gastroenterology.

65. Vankerckhoven V V A, T. V.; Geert, H.; Vancanneyt, M.; Swings, J.; Goossens, H. 2004. Establishment of the PROSAFE collection of probiotic and humna lactic acid bacteria. Microb Ecol Health Dis 16:131-136.

66. Rosander A, Connolly E, Roos S. 2008. Removal of antibiotic resistance gene-carrying plasmids from Lactobacillus reuteri ATCC 55730 and characterization of the resulting daughter strain, L. reuteri DSM 17938. Appl Environ Microbiol 74:6032-6040.

67. Cox G, Wright G D. 2013. Intrinsic antibiotic resistance: mechanisms, origins, challenges and solutions. Int J Med Microbiol 303:287-292.

68. Klein G, Hallmann C, Casas I A, Abad J, Louwers J, Reuter G. 2000. Exclusion of vanA, vanB and vanC type glycopeptide resistance in strains of Lactobacillus reuteri and Lactobacillus rhamnosus used as probiotics by polymerase chain reaction and hybridization methods. J Appl Microbiol 89:815-824.

69. Ammor M S, Florez A B, Mayo B. 2007. Antibiotic resistance in non-enterococcal lactic acid bacteria and bifidobacteria. Food Microbiol 24:559-570.

70. Klare I, Konstabel C, Werner G, Huys G, Vankerckhoven V, Kahlmeter G, Hildebrandt B, Muller-Bertling S, Witte W, Goossens H. 2007. Antimicrobial susceptibilities of Lactobacillus, Pediococcus and Lactococcus human isolates and cultures intended for probiotic or nutritional use. J Antmicrob Chemother 59:900-912.

71. Egervarn M, Danielsen M, Roos S, Lindmark H, Lindgren S. 2007. Antibiotic susceptibility profiles of Lactobacillus reuteri and Lactobacillus fermentum. J Food Prot 70:412-418.

72. Venugopal A A, Johnson S. 2012. Fidaxomicin: a novel macrocyclic antibiotic approved for treatment of Clostridium difficile infection. Clin Infect Dis 54:568-574.

73. Jaeger K E, Ransac S, Dijkstra B W, Colson C, van Heuvel M, Misset O. 1994. Bacterial lipases. FEMS microbiology reviews 15:29-63.

74. Kato T, Hayashi Y, Inoue K, Yuasa H. 2005. Glycerol absorption by Na+-dependent carrier-mediated transport in the closed loop of the rat small intestine. Biological & pharmaceutical bulletin 28:553-555.

75. Fujimoto N, Inoue K, Ohgusu Y, Hayashi Y, Yuasa H. 2007. Enhanced uptake of glycerol by butyrate treatment in HCT-15 human colon cancer cell line. Drug metabolism and pharmacokinetics 22:195-198.

76. De Weirdt R, Possemiers S, Vermeulen G, Moerdijk-Poortvliet T C, Boschker H T, Verstraete W, Van de Wiele T. 2010. Human faecal microbiota display variable patterns of glycerol metabolism. FEMS microbiology ecology 74:601-611.

77. Savidge T C, Pan W H, Newman P, O'Brien M, Anton P M, Pothoulakis C. 2003. Clostridium difficile toxin B is an inflammatory enterotoxin in human intestine. Gastroenterology 125:413-420.

78. Spinier J K, Brown A, Ross C L, Boonma P, Conner M E, Savidge T C. 2016. Administration of probiotic kefir to mice with Clostridium difficile infection exacerbates disease. Anaerobe 40:54-57.

1. An oral composition comprising cells of Lactobacillus reuteri (L. reuteri) pre-loaded with reuterin for use in the treatment or prevention of an infection caused by Clostridium difficile in an individual, wherein said oral composition is in the form of a tablet or a capsule.

2. The oral composition for use according to claim 1, wherein the composition is to be administered to said individual before, in combination with, and/or after a treatment with one or more antibiotic(s) effective against an infection caused by Clostridium difficile in said individual.

3. The oral composition for use according to any one of claim 1 or 2, wherein the treatment or prevention of the infection is accomplished without administering glycerol to the gastrointestinal tract in addition to the oral composition comprising cells of L. reuteri.

4. The oral composition for use according to any one of claims 1 to 3, wherein said L. reuteri is L. reuteri DSM 17938.

5. The oral composition for use according to any one of claims 1 to 4, wherein the tablet has a protective coating.

6. The oral composition for use according to any one of claims 1 to 4, wherein the capsule is an enteric coated capsule.

7. A method for the manufacture of an oral composition comprising L. reuteri pre-loaded with reuterin, comprising the steps of:
a) freeze-drying cells of L. reuteri pre-loaded with reuterin, and
b) distributing the freeze-dried cells into capsules or making tablets comprising the freeze-dried cells.

8. The method according to claim 7, wherein the tablet has a protective coating.

9. The method according to claim 7, wherein the capsule is an enteric coated capsule.

10. An oral composition obtained by a method according to any one of claims 7 to 9.

11. The oral composition according claim 10, further comprising an antibiotic(s) effective against an infection caused by Clostridium difficile.

12. The oral composition according to any one of claim 10 or 11, for use in the treatment or prevention of an infection caused by Clostridium difficile in an individual.

13. A method for the treatment or prevention of an infection caused by Clostridium difficile in an individual, said method comprising orally administering a composition comprising Lactobacillus reuteri (L. reuteri) cells pre-loaded with reuterin in a pharmaceutically effective amount to said individual.

14. The method according to claim 13, wherein said composition is administered to said individual before, in combination with, and/or after a treatment with one or more antibiotic(s) effective against an infection caused by Clostridium difficile in said individual.

15. The method according to any one of claim 13 or 14, wherein the treatment or prevention of the infection is accomplished without administering glycerol to the gastrointestinal tract in addition to the oral composition comprising cells of L. reuteri.

16. The method according to any one of claims 13 to 15, wherein said composition is as defined in any one of claims 10 to 12.


SEQUENCE LISTING (6 sequences)

SEQ ID NO: 1 - DNA, 22 bp, Artificial Sequence
  Primer
gcaggcctaacacatgcaagtc

SEQ ID NO: 2 - DNA, 20 bp, Artificial Sequence
  Primer
ctgctgcctcccgtaggagt

SEQ ID NO: 3 - DNA, 24 bp, Artificial Sequence
  Primer
ttgagcgatttacttcggtaaaga

SEQ ID NO: 4 - DNA, 21 bp, Artificial Sequence
  Primer
ccatcctgtactggctcacct

SEQ ID NO: 5 - DNA, 17 bp, Artificial Sequence
  Primer
cctacgggaggcagcag

SEQ ID NO: 6 - DNA, 18 bp, Artificial Sequence
  Primer
ccgtcaattcmtttragt